(19) United States
(12) Patent Application Publication
(10) Pub. No.: US 2008/0159908 A1
(43) Pub. Date: Jul. 3, 2008

(54) Method and apparatus for sterilizing indwelling catheters

(76) Inventor: Russell J. Redmond, Goleta, CA (US)

Correspondence Address: JAMES E. BRUNTON, ESQ., P. O. BOX 29000, GLENDALE, CA, 91209, US

(21) Appl. No.: 12/012,707

(22) Filed: Feb. 4, 2008

Related U.S. Application Data
(63) Continuation-in-part of application No. 11/517,963, filed on Sep. 9, 2006.

(51) Int. Cl. A61L 2/10

(52) U.S. Cl. 422/24

(57) ABSTRACT

A method and apparatus for delivering UV-C light energy to indwelling catheters by means of an elongated, flexible wand that is operably coupled with the UV-C light energy source. In one form of the invention the flexible wand is made from UV-C-transparent material and is fully disposable. In another form the wand is covered by a novel disposable cover made from UV-C-transparent materials and the wand is reusable.

This is a Continuation-In-Part of co-pending application Ser. No. 11/517,963 filed Sep. 9, 2006.

### BACKGROUND OF THE INVENTION

1. Field of the Invention

The present invention relates generally to irradiation devices for therapeutic purposes and the methods of using same. More particularly, the invention concerns a method and apparatus for sterilizing balloon-tipped endo-tracheal tubes, for sterilizing urinary drainage catheters and for treating urinary tract infections using ultraviolet radiation.

2. Discussion of the Prior Art

According to articles published by the Centers for Disease Control, in their Special Issue, “Emerging Infectious Diseases,” Vol. 7, No. 2, March-April 2001, “Nosocomial (hospital-acquired infections) bloodstream infections are a leading cause of death in the United States. Population-based surveillance studies of nosocomial infections in U.S. hospitals indicate a 5% attack rate or incidence of 5 infections per 1,000 patient-days.” With the advent of managed care and incentives for outpatient care, hospitals have a concentrated population of seriously ill patients, so rates of nosocomial infections are undoubtedly correspondingly higher.

By way of example, if 35 million patients are admitted each year to the approximately 7,000 acute-care institutions in the United States, the number of nosocomial infections—assuming overall attack rates of 2.5%, 5%, or 10%—would be 875,000, 1.75 million, or 3.5 million, respectively. If 10% of all hospital-acquired infections involve the bloodstream, 87,500, 175,000 or 350,000 patients acquire these life threatening infections each year. These are staggering numbers, especially considering this is a problem that the patients did not have when they entered the hospital.

With the forgoing in mind, ultraviolet (UV) light has long been used for disinfection and sterilization. In recent years, the widespread availability of low to medium pressure mercury bulbs has led to the development of devices which use UV-C for air purification and to decontaminate water supplies. UV-C has also found some limited use in food processing and in medical device sterilization. UV-C is a high frequency wavelength of light within the ultraviolet band and has been shown to be the most bactericidal type of ultraviolet light.

By way of background, UV light consists of high energy photons, which occupy the 200 to 400 nanometer wavelengths of the electromagnetic spectrum. This means that UV light emits slightly less energy than soft X-ray radiation, but significantly more than visible light. UV energy does not directly kill pathogens, but rather causes a photochemical reaction within the genetic structure which inhibits the ability of the pathogens to reproduce, therefore, in effect, killing the pathogen.

The amount of energy delivered by UV light is inversely proportional to its wavelength; therefore, the shorter the wavelength, the greater the energy produced. In general, the UV light portion of the spectrum is made up of three segments; UV-A (315-400 nm), used for sun-tanning lamps, UV-B (280-315 nm) and UV-C (200-280 nm). The UV-B and, as previously mentioned, UV-C regions contain wavelengths with the best germicidal action. Studies have shown that the wavelengths most effective in killing microbes are between 250-265 nm. This value corresponds nicely with the light energy output of a typical, commercially available UV-C germicidal lamp, which produces most of its energy output in the range of 254 nm.

An application of particular interest for the apparatus of the present invention concerns the sterilization of endo-tracheal tubes (ET tubes). In this regard, many patients admitted to the intensive care unit (ICU) need mechanical assistance to help them breathe properly during treatment and recuperation. Typically, an ET tube having a distal balloon is placed within the patient's trachea. This tube is then connected to an external ventilator to artificially inflate and deflate the patient's lungs. The purpose of the ET tube's distal balloon is to not only maintain the tube in proper position, but also to help prevent sub-glottic secretions from passing into the lungs as these secretions often are contaminated with bacteria. The annular space between the outer surface of the tube and the inner wall of the trachea proximal to the balloon creates a breeding ground for bacteria as the sub-glottic secretions collect above the balloon seal. These, potentially infected, secretions, are periodically aspirated by the nursing staff via a suction catheter attached to the ventilator line. Unfortunately, the balloon often has an imperfect seal and infected secretions can track past the balloon into the patient's lung's where they can colonize, multiply and frequently result in nosocomial pneumonia called Ventilator-Associated Pneumonia (VAP).

Bacteria can also be introduced within the lumen of the endo-tracheal tube via leakage of oral fluids, nursing contamination during aspiration or even during initial ventilator set-up. This internal contamination can also lead to bacterial infection of the endo-tracheal tube's lumen and VAP, when the bacterial colonies break off and move into the lungs via the normal movement air during ventilation.

Once bacteria have contaminated the inner and/or outer surfaces of a synthetic indwelling catheter, micro-biological research has demonstrated that a very difficult to kill bacterial ‘biofilm’ is created on the catheter's surface. This biofilm resists anti-biotic treatment as it forms a protective barrier over the bacterial colony and reduces the effectiveness of the pharmacological treatment. This is especially problematic for VAP, as the patient is unable to breathe on their own, so the infected endo-tracheal tube must be replaced with a new, sterile ET tube. Unfortunately, the new ET tube is placed in the same infected location within the trachea, simulating new biofilm formation and possible new infection.

The Centers for Disease Control and Prevention has stated that VAP accounts for approximately 15-27% of all infections acquired in the medical ICU and CCUs. It is the second most common hospital-associated infection (after urinary tract infection). It is also recognized as one of the leading killers among hospital-acquired infections, causing an estimated 26,000 deaths every year in the United States alone.

Another application of particular interest for apparatus of the present invention concerns the sterilization of balloon-tipped urinary drainage catheters. In this regard the placement into the patient of a balloon-tipped urinary drainage catheter in the hospital, clinic or nursing home provides a significant opportunity for bacteria to colonize the inner lumen of the catheter or the annular space between the outer surface of the catheter and the inner wall of the urethra. Although antibiotic treatment for urinary tract infections has been successful in the past, such treatment is complicated by the need to maintain artificial drainage during treatment and may be further complicated by the increase in the incidence of Methacillin Resistant Staph Aureus (MRSA) infections in the hospital and in the community at large.

### SUMMARY OF THE INVENTION

It is an object of the present invention to provide a novel hand-held UV radiation device that is particularly effective in treating bacterial, viral, fungal and parasitic infections found in several of the body's anatomical orifices.

It is another object of the present invention to provide a device of the aforementioned character that is particularly effective in treating MRSA colonies in the nose and on the skin surface of a patient.

Another object of the invention is to provide a device of the character described in the preceding paragraph that can eliminate, or greatly reduce, the MRSA colonies without significantly damaging the underlying tissue of the patient.

Another object of the present invention is to provide a novel hand-held radiation device that is effective in eliminating, or at least significantly reducing, the severity of certain viral infections such as avian influenza.

Another object of the present invention to provide a method and apparatus for delivering UV-C light energy to indwelling catheters by means of an elongated, flexible wand that is operably coupled with the UV-C light energy source.

Another object of the invention is to provide an apparatus of the aforementioned character in which the flexible wand is made from UV-C-transparent material and is fully disposable, or alternatively, when used in connection with a novel disposable cover made from UV-C-transparent materials, such as polypropylene or polyethylene, is reusable.

Another object of the invention is to provide an apparatus as described in the preceding paragraph in which the UV-C light energy source is mounted within a handheld instrument having an integral switch and timer and to which the flexible wand is interconnected and is either battery-operated, or alternatively, is fitted with an AC Adapter that would permit the light source to be connected to a standard electrical outlet.

Another object of the invention is to provide an apparatus as described in the preceding paragraphs in which the handheld instrument has an integral interlock switch that prevents actuation of the instrument unless a Wand Cover, or alternatively, a disposable Wand shaft is in proper engagement with the instrument.

Another object of the invention is to provide an apparatus of the character described in the preceding paragraphs in which the UV-C light energy is guided along the wand by means of a multiplicity of optical fibers.

Another object of the invention is to provide an apparatus of the character described in the preceding paragraphs in which the UV-C light energy is guided along the wand by means of a liquid-filled light-guide.

Another object of the invention is to provide an apparatus of the character described in the preceding paragraphs in which the UV-C light energy is generated at the tip of the flexible wand by a plurality of light emitting diodes that are electrically coupled with the handheld instrument by a plurality of small conductor wires.

Another object of the invention is to provide an apparatus for delivering UV-C light into the lumen of a balloon-tipped endo-tracheal tube.

Another object of the invention is to provide an apparatus for delivering UV-C light to the exterior surface of a balloon-tipped endo-tracheal tube.

Another object of the invention is to provide an apparatus for delivering UV-C light into the lumen of a urinary drainage catheter.

Another object of the invention is to provide a sterilization apparatus, of the type described in the preceding paragraphs, that can be used by caregivers with a minimum amount of training.

Another object of the invention is to provide a sterilization apparatus of the character described which is of simple construction and can be inexpensively produced.

### BRIEF DESCRIPTION OF THE DRAWINGS

FIG. 1 is a generally perspective view of one form of the irradiation treatment device of the present invention.

FIG. 2 is an enlarged, fragmentary, perspective view of the area designated in FIG. 1 as “2”.

FIG. 3 is an enlarged, generally perspective view of one form of a disposable probe cover usable with the apparatus shown in FIG. 1 of the drawings.

FIG. 4 is a generally perspective, foreshortened, exploded view of the device illustrated in FIG. 1, here shown interconnected with an energy source and depicted with the disposable probe cover in position over the forward portion of the device.

FIG. 5 is a generally schematic view illustrating the interconnection of the various operating components of the device shown in FIG. 1.

FIG. 6 is a generally diagrammatic view of an alternate form of the sterilization apparatus of the present invention for use in sterilizing indwelling catheters and is there shown in use for the sterilization of the lumen of an endo-tracheal tube.

FIG. 7 is a generally schematic view illustrating the interconnection of the various operating components of the device shown in FIG. 6.

FIG. 7A is a generally schematic view, similar to FIG. 7, but showing the wand portion of the device disengaged from the handheld housing portion thereof.

FIG. 8 is a generally diagrammatic view of still another form of the sterilization apparatus of the present invention for use in sterilizing indwelling catheters.

FIG. 9 is a generally perspective view of still another form of the sterilization apparatus of the invention that is similar to the apparatus shown in FIG. 6, but includes a reusable, flexible wand and a disposable wand cover.

FIG. 9A is a greatly enlarged, cross-sectional view of the area designated in FIG. 9 as “9A”.

FIG. 10 is a greatly enlarged, generally perspective, fragmentary view of the area designated in FIG. 9 by the numeral 10.

FIG. 10A is a greatly enlarged, fragmentary side-elevational view, partly in cross-section, showing the wand cover of FIG. 9 interconnected with the handheld housing portion of FIG. 9.

FIG. 11 is a greatly enlarged, generally perspective view of the area designated in FIG. 9 by the numeral 11, but shown rotated by 180° to better illustrate the configuration of the end portion of the wand cover.

FIG. 12 is a generally perspective, diagrammatic view illustrating the embodiment of the invention shown in FIG. 6 in position within the annular space between the outside of the endo-tracheal tube and the wall of the trachea.

FIG. 13 is a generally perspective, diagrammatic view illustrating the embodiment of the invention shown in FIG. 6 in position for insertion into the lumen of a urinary drainage catheter.

FIG. 14 is a generally perspective, diagrammatic view, similar to FIG. 13, but showing the flexible wand inserted into the lumen of a urinary drainage catheter.

### DISCUSSION OF THE INVENTION

### Definitions

As used herein the following terms have the following meanings:

“UV-C” means ultraviolet light having a wavelength of between about 200 and about 280 nm.

“Wand” means a small flexible shaft that can be introduced into the intra-luminal spaces of indwelling catheters and can be coupled with a reusable instrument having an integral switch and timer. The wand can either be fully disposable or reusable through the use of a disposable cover and can be used to deliver UV-C light energy into the lumen of indwelling catheters. The light source can be mounted within the instrument handle and guided along the wand by various means, including flexible optic light bundles and a liquid-filled light-guide. Additionally, the UV-C light energy can be generated at the tip of the flexible wand by a plurality of light emitting diodes that are electrically coupled with the handheld instrument by a plurality of small conductor wires.

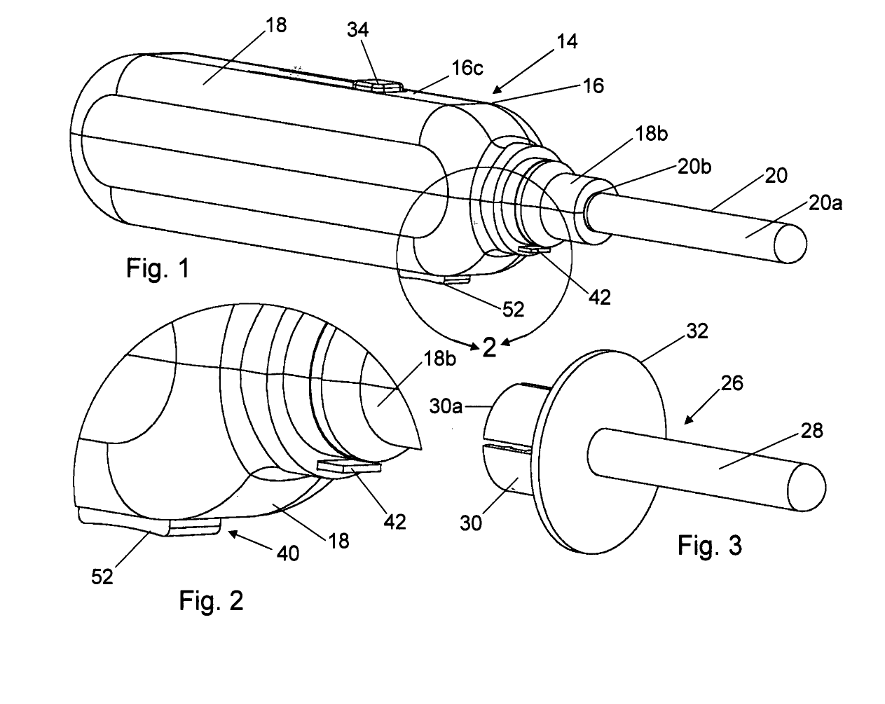

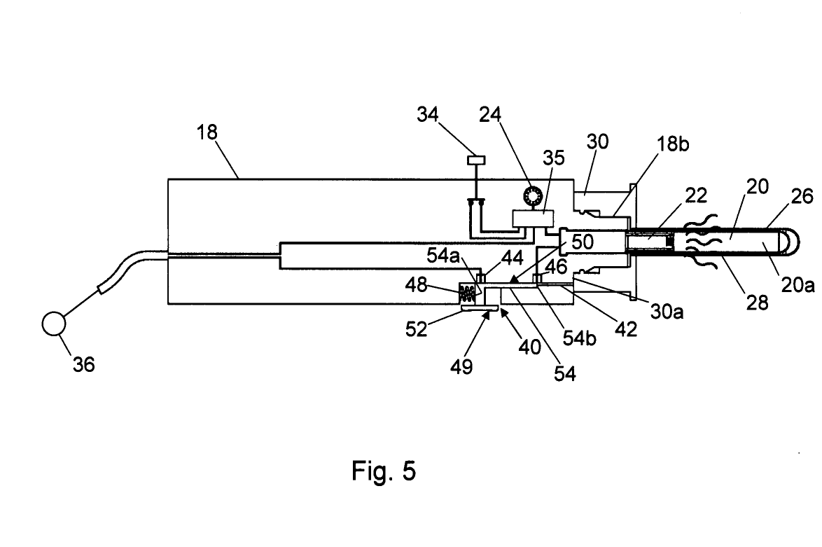

Referring to the drawings and particularly to FIGS. 1 through 5, one form of the novel irradiation device of the invention for treating bacterial colonies on the skin of the patient and in various body orifices, such as the mouth, the urethra, the vagina, the rectum and the external ear canal is there shown and generally designated by the numeral 14. This form of the device here comprises a housing 16 having a hollow, generally cylindrical-shaped body portion 18 and an elongated probe portion 20 that is connected to housing portion 18 and extends outwardly there from. Probe portion 20, which has a forward irradiating portion 20a and rearward portion 20b, is connected to the forward hub portion 18b of body portion 18 in the manner shown in FIGS. 1 and 5. Operably associated with probe 20 is a conventional radiation source 22 for emitting UV radiation in a direction toward the elongated probe portion 20a. Disposed within housing 16 is a timer 24, the function of which will presently be described (FIG. 5).

Removably connected to the forward irradiating portion of the elongated probe is a uniquely constructed, substantially UV radiation-transparent probe cover 26. As best seen in FIG. 3, probe cover 26 includes a generally tubular-shaped forward portion 28 that covers the forward portion of the probe and a generally tubular-shaped, slotted-connector portion 30 that is telescopically receivable over the forward hub portion 18b of housing portion 18. Forward portion 28 can be constructed from polypropylene, certain varieties of Teflon that are transparent to UV light in the UV-C range (200-280 nm) and other amorphous fluropolymers. Disposed intermediate forward portion 28 and connector portion 30 is an enlarged diameter radiation shield 32 that functions to shield the user's hand from radiation.

As indicated in FIG. 3, shield 32 is constructed from a UV radiation-opaque material, such as polycarbonate, polystyrene and like materials that will effectively shield the surrounding tissue so that only the target areas of the patient will receive the germicidal UV energy.

It is to be understood that the unique design of the probe cover as shown in the drawings not only provides protection for the UV light probe 20 and a method of preventing probe contamination, the probe also sets the proper distance from the light source to the tissue being exposed to the UV radiation. This factor is especially important because the energy transmitted to the tissue decreases exponentially with the distance between the light source and the tissue surface. In this regard, it should be appreciated that in carrying out the method of the invention for treating bacterial, viral, fungal, and parasitic infections in the patient's tissue, pre-setting the distance between light source and tissue in this manner is quite different than simply holding a UV light over the tissue as this approach does not stretch the tissue as does the UV light probe 20. In accordance with one form of the method of the invention, the tissue is uniformly stretched so that the tissue is uniformly exposed to the light energy which is critical to the successful germicidal use of UV light as it is a ‘line of sight’ process. Creases or folds can hide bacteria from exposure to the light energy, preventing them from receiving a lethal dose of irradiation.

Additionally, the probe covers of the apparatus are designed so that they slightly dilate the lumen of the orifice into which they are inserted, such as the vagina and rectum. In accordance with the method of the invention, this dilation sets the distance from light source to tissue surface very accurately as the dilated tissue conforms to the probe cover, giving uniform coverage of the UV light energy to the tissue to be treated.

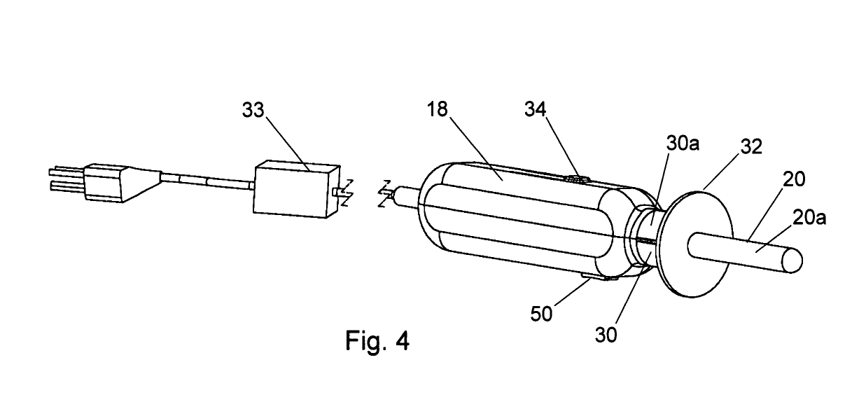

Carried by housing 16 is switching means for controllably energizing the source of UV radiation 22, which here comprises a conventional, readily commercially available UV-generating lamp. In the present form of the invention this switching means, which comprises a part of the electrical circuitry of the invention (see FIG. 5), is provided in the form of a conventional, readily commercially available, one-touch activator switch 34. Switch 34 is mounted on the exterior wall 16c of the housing 16 (FIG. 1) and, as shown in FIGS. 4 and 5, is operably interconnected in a conventional manner with a source of electrical power 36 and with the UV radiation source 22 via a conventional transformer 33, a conventional relay 35, the timer 24 and the novel disabling means of the invention, the character of which will presently be described. Timer 24, which ensures that the tissue is irradiated for a predetermined period of time, is here provided in the form of a conventional, readily commercially available, adjustable timer that can be pre-set to control the length of time that the UV radiation source remains energized after the circuit is closed. Radiation source 22, timer 24, switch 34, relay 35 and transformer 33 are interconnected within the electrical circuitry in a manner well understood by those skilled in the art.

An important feature of the present invention is the provision of a novel combination cover-ejecting means and safety-disabling means. The cover-ejecting means permits the operator to safely eject the cover after it becomes contaminated without touching the cover. The novel safety-disabling means here functions to prevent energizing the UV radiation source by the switching means of the invention unless the probe cover 26 is correctly in position over the probe 20. In the present form of the invention this novel combination cover-ejecting means and safety-disabling means comprises an assembly generally designated by the numeral 40 that is carried by housing 16 in the manner best seen in FIGS. 2 and 5. In this embodiment of the invention, assembly 40 comprises an ejector member 42 that is movable by portion 30 of the cover 20 from a first extended position shown in FIG. 2 to a second retracted position shown in FIG. 5.

With the ejector member having been moved by the rim portion 30a of the cover (see FIGS. 3, 4 and 5) into the second retracted position shown in FIG. 5, the electrical circuit between points 44 and 46 is completed so that the source of radiation can be energized by the operation of switch 34. Once energized, the UV radiation source will remain in its energized state for the period of time set on the timer 24. At the expiration of this time, the UV energy source will be automatically de-energized and the probe cover can be safely ejected from the hub portion 18b of the housing.

It is to be noted that as the probe cover is ejected from the hub portion 18b of the housing, the ejector member is urged by the biasing means, or spring 48, into the extended position shown in FIG. 2, thereby breaking the electrical circuit between points 44 and 46 so that the source of radiation 22 cannot be re-energized by the operation of switch 34.

On the other hand, as can be seen by referring to FIGS. 2 and 5, movement of the ejector member toward its retracted position by positioning the cover 26 over the probe 20 in the manner shown in FIGS. 4 and 5, will once again complete the electrical circuit between points 44 and 46.

Movement of the ejector member into the retracted position shown in FIG. 5 will also move a finger-engaging ejector slide assembly 49, which is slidably connected to housing 16, rearwardly of the housing against the urging of spring 48. Finger-engaging ejector slide assembly 49, which comprises a part of the cover-ejecting means of the invention, includes a finger-engaging ejector slide 52 and an integrally formed ejector frame 54. Portion 54a of ejector frame 54 is in operable engagement with spring 48, while portion 54b of the frame is in operable engagement with ejector member 42. With this construction, after the irradiation treatment has been completed, the contaminated cover can be readily ejected from the hub portion 18b by merely sliding the finger-engaging ejector slide 52 forwardly of the housing. In this regard, as the finger-engaging ejector slide 52 moves forwardly, the ejector member 42 will be caused to move toward its extended position and, in so doing, will safely eject the contaminated cover from the hub portion 18b of the housing. In this way, the operator can safely and conveniently eject the contaminated cover into an appropriate disposal container without touching it.

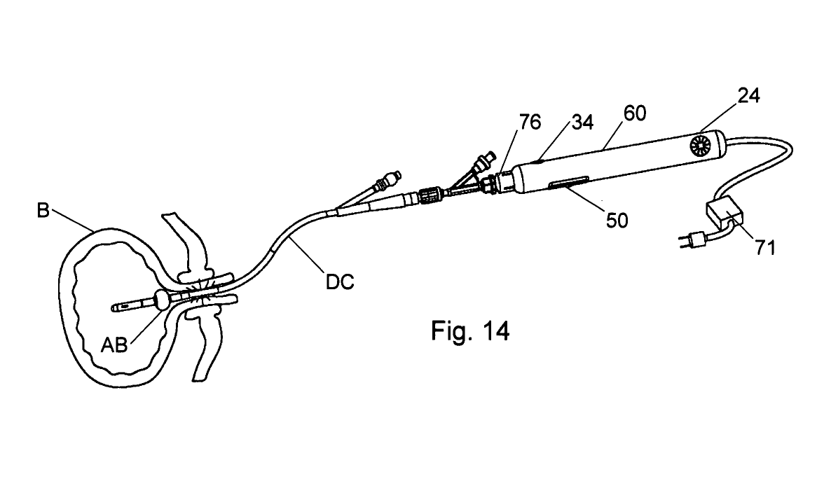

In using the device of the invention to carry out the method of the invention, the user first positions a clean probe cover 26 over the probe in the manner shown in FIGS. 4 and 5. This done, timer 24 is set and the tip portion of the cover is inserted into the body orifice, such as the patient's urinary drainage tube, in the manner illustrated in FIG. 14 for a timed application to the urethra of germicidal UV energy.

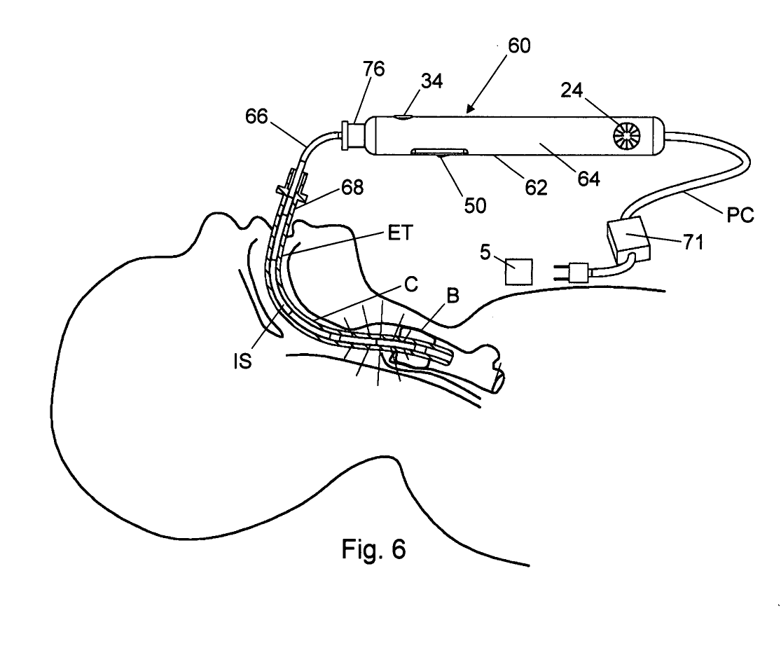

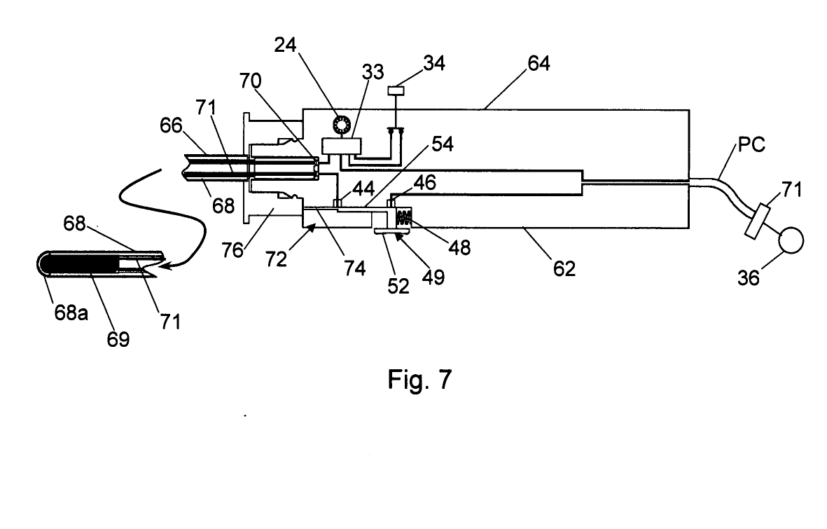

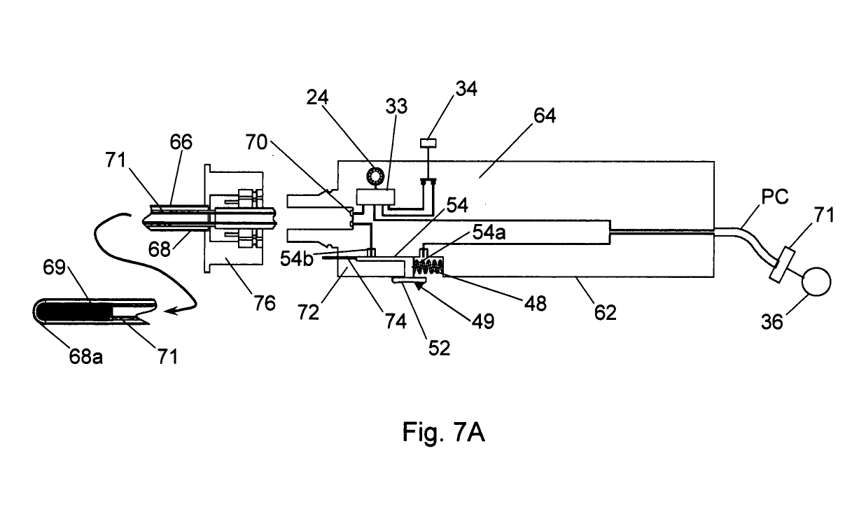

Turning next to FIGS. 6, 7 and 7A of the drawings, an alternate form of the sterilization apparatus of the invention is there shown and generally designated by the numeral 60. This form of the invention is similar in many respects to that previously described and like numerals are used in FIGS. 6, 7 and 7A to identify like components. As illustrated in FIGS. 6 and 7 of the drawings, the sterilization apparatus of this latest form of the invention, which is specially designed for exposing an indwelling catheter “C” to germicidal ultraviolet light here comprises a generally torpedo-shaped housing 62 having a hollow, generally cylindrical-shaped body portion 64 and an elongated, flexible wand 66 connected to the housing. It is to be understood that, while the shape of the handheld instrument housing is slightly different from that shown in FIGS. 1 through 5 of the drawings, the electrical circuitry that is housed within the housing 64 is quite similar in construction and operation to that described in connection with the embodiment of FIGS. 1 through 5.

Wand 66 here comprises a small flexible shaft 68 that, as shown in FIGS. 6 and 7, can be introduced into the internal passageway, or intra-luminal space “IS” of the indwelling catheter and can be coupled with the reusable housing 64 in the manner presently to be described.

Wand 66 can either be fully disposable or, in a manner presently to be described, can be reusable if desired. In the embodiment of the invention shown in FIGS. 6, 7 and 7A, the UV-C light energy is generated at the tip 68a of the flexible shaft 68 by a plurality of light emitting diodes 69 that are electrically coupled with and energized via an electrical contact 70 carried within housing 62 by a plurality of small conductor wires 71 (see FIG. 7).

Also carried by housing 64 is a switching means for controllably energizing the source of germicidal UV-C radiation, or light emitting diodes 69, and a timer 24 that is substantially identical in construction and operation to the timer previously described in connection with the embodiment of FIGS. 1 through 5. The switching means of this latest form of the invention, which comprises a part of the electrical circuitry of the invention, is also substantially identical in construction and operation to that previously described and is here provided in the form of a conventional, readily commercially available, one-touch activator switch, or button 34. Switch 34 is mounted on the exterior wall of the housing 62 (FIG. 6) and, as shown in FIG. 7, is interconnected with the UV-C radiation source, or light emitting diodes 69 via contact 70 and with a source of electrical power 36 via a conventional power cord “PC” and a conventional, readily commercially available AC adapter 71. Switch 34 is also operably interconnected in the manner shown in FIG. 7 of the drawings with a conventional relay 33, with a timer 24 and with the novel disabling means of the invention, the character of which will presently be described.

An important feature of this latest form of the invention is the provision of a novel combination wand ejection means and safety-disabling means. The wand ejection means is similar in construction and operation to the previously described cover-ejecting means and permits the operator to safely eject the wand after it becomes contaminated without touching the wand. The novel safety-disabling means here functions to prevent energizing the electrical contact 70 by the switching means of the invention unless the wand is correctly connected to the housing. In the present form of the invention this novel combination wand-ejecting means and safety-disabling means comprises an assembly generally designated by the numeral 72 that is carried by housing 62 in the manner best seen in FIG. 7. In this latest embodiment of the invention, assembly 72 comprises an ejector member 74 that is movable by hub portion 76 of the wand from a first extended position shown in FIG. 7A to a second retracted position shown in FIG. 7.

With the ejector member having been moved by portion 76 into the second retracted position shown in FIG. 7, the electrical circuit between points 44 and 46 is completed so that the source of radiation can be energized by the operation of switch 34. Once energized, the UV radiation source will remain in its energized state for the period of time set on the timer 24. At the expiration of this time, the UV energy source will be automatically de-energized and the wand can be safely ejected from the reusable, handheld housing.

An important feature of the apparatus of this latest form of the invention is switch interlock means that is carried by housing 62 for preventing energization of the electrical contact 70 when the wand is ejected. In this regard it is to be noted that as the wand is ejected, the ejector means, or member 74 which is also carried by housing 62 is urged by the biasing means, or spring 48, into the extended position shown in FIG. 7A, thereby breaking the electrical circuit between points 44 and 46 so that the electrical contact 70 cannot be re-energized by the operation of switch 34.

On the other hand, as can be seen by referring to FIGS. 7 and 7A, movement of the ejector member toward its retracted position by hub portion 76 of the wand in the manner shown in FIG. 7 will once again complete the electrical circuit between points 44 and 46 and permit the light emitting diodes 69 to be energized.

Movement of the hub portion 76 of the wand assembly into the retracted position shown in FIG. 7 will also move a finger-engaging ejector slide assembly 49, which is slidably connected to housing 64, rearwardly of the housing against the urging of spring 48. Finger-engaging ejector slide assembly 49, which comprises a part of the wand-ejecting means of the invention, includes a finger-engaging ejector slide 52 and an integrally formed ejector frame 54. Portion 54a of ejector frame 54 is in operable engagement with spring 48, while portion 54b of the frame is in operable engagement with ejector member 74. With this construction, after the irradiation treatment has been completed, the contaminated wand assembly can be readily ejected from the handheld instrument body 64 by merely sliding the finger-engaging ejector slide 52 forwardly of the housing. In this regard, as the finger-engaging ejector slide 52 moves forwardly, the ejector member 74 will be caused to move toward its extended position and, in so doing, will safely eject the contaminated wand assembly from the housing. In this way, the operator can safely and conveniently eject the contaminated wand assembly into an appropriate disposal container without touching it.

Referring particularly to FIG. 6 of the drawings, the apparatus of the invention is there shown being used in conjunction with a balloon-tipped endo-tracheal tube (ET). In this regard, many patients admitted to the intensive care unit of the hospital need mechanical assistance to help them breathe properly during treatment and recuperation. To assist the patient's breathing, a balloon-tipped endo-tracheal tube is typically placed within the patient's trachea. The tube is then connected to an external ventilator to artificially inflate and deflate the patient's lungs.

In using the device of the invention to carry out the method of the invention as illustrated in FIG. 6 of the drawings, the timer 24 is first set to the desired irradiation time and the flexible wand is inserted to a known depth within the indwelling catheter “C” so that the tip 68a thereof is proximal to the inflated balloon “B” (see FIG. 6). Once the wand is in position within the catheter, the light emitting diodes 69 are actuated by the switching means, or switch 34 and the timed UV-C light cycle is directed to the inner walls of the catheter. With proper power settings and proper catheter material selection, sufficient UV-C energy can be generated within the catheter lumen so that the UV-C energy will to pass through the catheter walls and treat the external surface of the catheter as well as the inner surface of the passageway thereof.

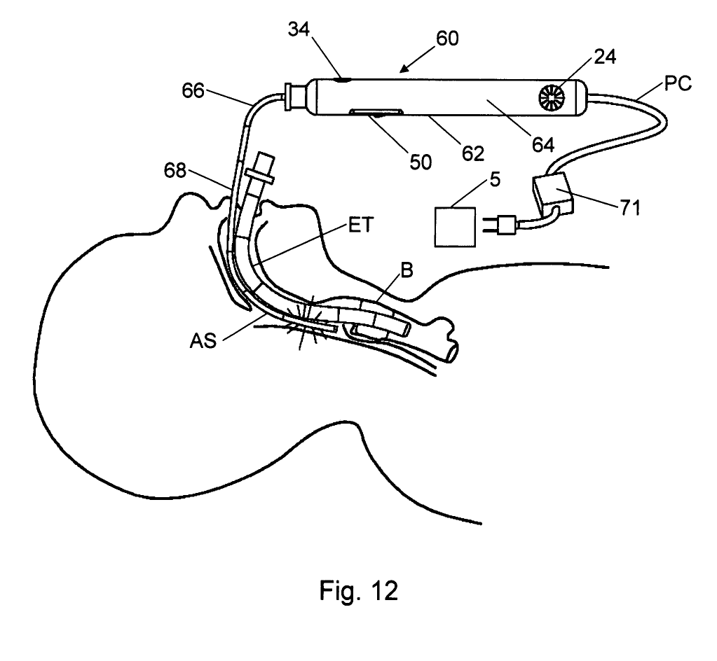

If the wand 66 is used in conjunction with a catheter that is UV-C-opaque, the initial treatment will only occur on the inner surfaces of the catheter. This requires the user to treat the outer surfaces of the catheter in a manner presently to be described and as illustrated in FIG. 12 of the drawings by repositioning the flexible wand along the outer endo-tracheal catheter surface, between the ET-tube and the wall of the trachea.

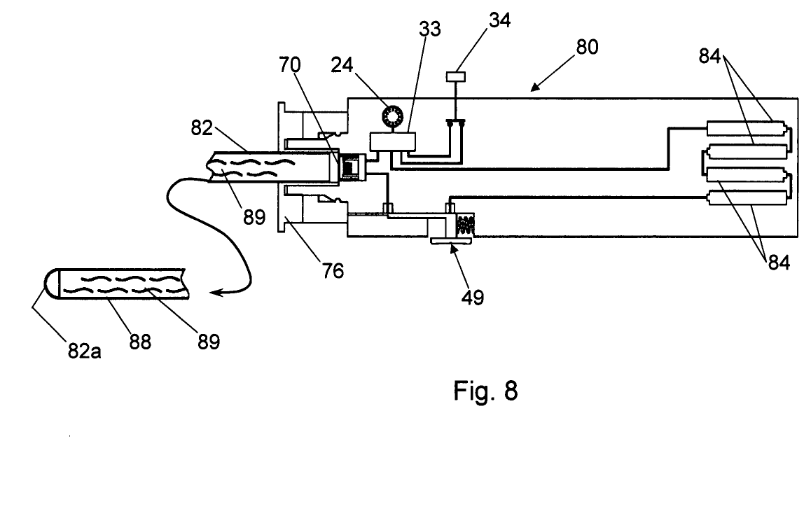

Referring now to FIG. 8 of the drawings, still another form of the sterilization apparatus of the invention is there shown and generally designated by the numeral 80. This form of the invention is similar in many respects in both construction and operation to that previously described and like numerals are used in FIG. 8 to identify like components. This latest form of the invention, like the earlier described embodiments, is specially designed for exposing an indwelling catheter “C” to germicidal ultraviolet light. The primary differences between the embodiment of the invention shown in FIG. 8 and the embodiment of the invention shown in FIGS. 6, 7 and 7A resides in the different energy source for energizing the germicidal ultraviolet light and the different differently configured flexible wand assembly 82.

With regard to the energy source for energizing the germicidal ultraviolet light, the energy source here comprises a plurality of conventional, readily commercially available dry cell batteries 84 that are housed within the slightly differently configured handheld instrument housing 86.

With regard to the wand assembly 82, once again the wand assembly of this latest form of the invention comprises an elongated, small flexible shaft 88 that can be introduced into the intra-luminal space “IS” of an indwelling catheter such is that shown in FIG. 6 of the drawings. However, catheter assembly 82 here comprises a liquid-filled light-guide 89 that is housed within flexible shaft 88 in a manner shown in FIG. 8 of the drawings.

In using the device of this latest form of the invention to carry out the method of the invention as illustrated in FIG. 6 of the drawings, the timer 24 is first set to the desired irradiation time and the flexible wand 82 is inserted to a known depth within the indwelling catheter “C” so that the tip 82a thereof is proximal to the inflated balloon “B” (see FIGS. 6 and 8). Once the wand is in position within the catheter, a UV lamp 91 is actuated by the switch 34, which interconnects the light source with the batteries 84. With the UV lamp thusly actuated, the timed UV-C light cycle is directed to the inner walls of the catheter. With proper power settings and proper catheter material selection, sufficient UV-C energy can be generated within the catheter lumen so that the UV-C energy will to pass through the catheter walls and treat the outer surface of the catheter as well as the inner surface thereof.

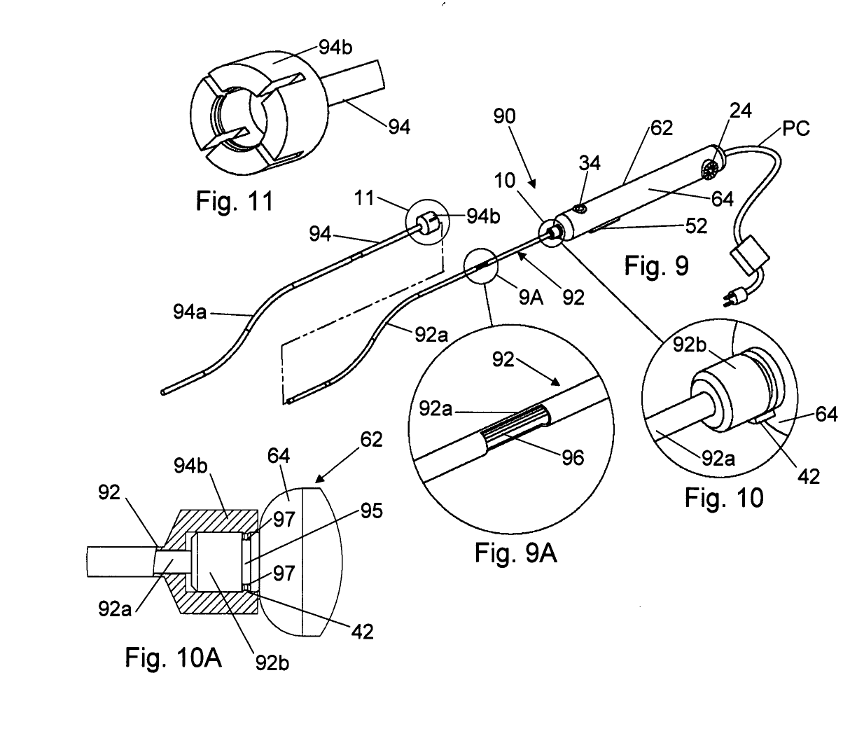

Turning next to FIGS. 9, 10, 10A and 11, yet another form of the sterilization apparatus of the invention is there shown and generally designated by the numeral 90. This form of the invention is also similar in many respects in both construction and operation to the previously described embodiments and like numerals are used in FIGS. 9, 10, 10A and 11 to identify like components. This latest form of the invention, like the earlier described embodiments, is specially designed for exposing an indwelling catheter “C” to germicidal ultraviolet light. The primary differences between the embodiment of the invention shown in FIG. 8 and the embodiment of the invention shown in FIGS. 6, 7 and 7A resides in the differently configured flexible wand assembly 92 and the use of wand cover 94 that includes a UV-C-transparent disposable protective sheath 94a that is receivable over the elongated shaft 92a of the wand assembly 92. As before, the elongated shaft 92a can be introduced into the intra-luminal space “IS” of an indwelling catheter such as that shown in FIG. 6 of the drawings.

With regard to the flexible wand assembly 92, this novel, reusable assembly here comprises a multiplicity of optical fibers 96 that are housed within the elongated shaft 92a of the wand assembly 92 (see FIG. 9A). As shown in FIGS. 9 and 10, flexible wand assembly 92 also includes a connector hub 92b to which the elongated shaft 92 of the wand assembly is interconnected. As illustrated in FIGS. 9, 10 and 10A of the drawings connector hub 92b is fixedly connected to the body portion 64 of the hand held instrument 62. For a reason presently to be described, connector hub 92b is provided with a circumferentially extending groove 95.

As illustrated in FIGS. 9 and 11, disposable cover assembly 94 includes a slotted-connector hub 94b that is close to receivable over the connector hub 92b of the wand assembly in the manner shown in FIG. 10A. To hold the removable disposable cover assembly 94 in position relative to connector hub 92b, the connector hub 94b is provided with a plurality of circumferentially spaced, yieldably deformable fingers 97 that are receivable within the groove 95 formed in connector hub 92b.

With the construction described in the preceding paragraphs and illustrated in FIGS. 9 through 11, after the irradiation of the indwelling catheter has been completed in the manner previously described, the contaminated cover assembly 94 can be readily ejected from the handheld instrument body 64 by merely sliding the finger-engaging ejector slide 52 forwardly of the housing. In this regard, as the finger-engaging ejector slide 52 moves forwardly, the ejector member 74 will be caused to move toward its extended position shown in FIG. 10 and, in so doing, will safely eject the contaminated wand assembly from the housing. In this way, the operator can safely and conveniently eject the contaminated wand assembly into an appropriate disposal container without touching it.

In using the device of this latest form of the invention to carry out the method of the invention as illustrated in FIG. 1 of the drawings, the timer 24 is first set to the desired irradiation time and the elongated shaft 92 of the flexible wand, along with the protective cover 94a that covers the shaft 92, is inserted to a known depth within the indwelling catheter “C” so that the tip of the wand is proximal to the inflated balloon “B” (see FIG. 6). Once the wand is in position within the catheter, the UV lamp is actuated by the switch 34, which interconnects a light source, such as lamp 91, with the source of electricity. In this regard, it is to be understood that the electrical circuitry that is housed within housing 64 is substantially identical in construction and operation to that illustrated in FIG. 8 of the drawings and earlier described herein. With the UV lamp actuated, the timed UV-C light cycle is directed to the inner walls of the catheter. With proper power settings and proper catheter material selection, sufficient UV-C energy can be generated within the catheter lumen so that the UV-C energy will pass through the catheter walls and treat the outer surface of the catheter as well as the inner surface thereof. Following in the irradiation of the catheter, the protective cover assembly 94 can be ejected from the hub 92b in the manner previously described and appropriately discarded. Because the elongated shaft of the wand has been protected by the cover assembly 94, the wand assembly can be reused after it is once again covered with a new protective covering 94.

Referring to FIG. 12 of the drawings, the embodiment of the invention illustrated in FIG. 6 is there shown in position within the annular space “AS” between the outside of the endo-tracheal tube ET and the wall of the trachea. As previously mentioned, if the wand 68 is used in conjunction with a catheter that is UV-C-opaque, the initial treatment will only occur on the inner surfaces of the catheter. This requires the user to treat the outer surfaces of the catheter in the manner illustrated in FIG. 12 by repositioning the flexible wand along the outer endo-tracheal catheter surface, between the ET-tube and the wall of the trachea. This repositioning step is somewhat similar to the placement of an aspiration/suction catheter, which is a routine nursing maneuver in the treatment of ventilated patients.

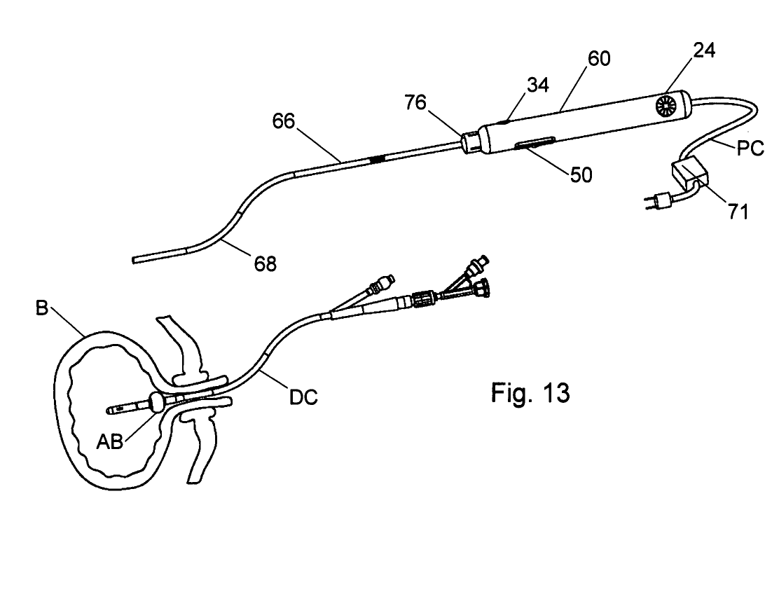

Turning next to FIGS. 13 and 14 of the drawings, these drawings illustrate the use of the apparatus of the invention for irradiating an insitu urinary drainage catheter “DC” of a character well understood by those skilled in the art and having a distal anchoring balloon “AB”. FIG. 13 illustrates the apparatus of the invention, which is of the character illustrated in FIG. 7 of the drawings, in position to be inserted into a urinary drainage catheter “DC” that is in position within the bladder “B” of the patient. FIG. 14 of the drawings illustrates the appearance of the apparatus after the elongated, flexible wand has been inserted into the urinary drainage catheter.

The apparatus of the invention can also be used for treating urinary tract infections. In this use of the apparatus it is necessary to initially, temporarily remove an infected urinary drainage catheter “DC” of the character constructed from UV-C-opaque materials from the patient. After this is accomplished, the flexible wand can be inserted directly into the urethra to effectively treat infection of the urethral tissue.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

We claim:

1. An apparatus for exposing an indwelling catheter to germicidal ultraviolet-see light comprising:
(a) a source of germicidal UV-C light; and
(b) an elongated, flexible wand connected to said source of germicidal UV-C light for delivering the UV-C light to said indwelling catheter.

2. The apparatus as defined in claim 1 in which said flexible wand comprises a multiplicity of optical fibers.

3. The apparatus as defined in claim 1 in which said flexible wand comprises a liquid-filled light-guide.

4. The apparatus as defined in claim 1 in which said flexible wand includes a distal end having affixed thereto at least one light emitting diode.

5. The apparatus as defined in claim 1 in which said apparatus further includes a UV-C-transparent disposable wand cover for covering said elongated, flexible wand.

6. The apparatus as defined in claim 1 in which said indwelling catheter includes an internal passageway and in which said elongated, flexible wand is telescopically receivable within said internal passageway of said indwelling catheter.

7. The apparatus as defined in claim 1 in which said indwelling catheter comprises an internal passageway and an external wall and in which said elongated, flexible wand is movable along said external wall of said indwelling catheter.

8. The apparatus as defined in claim 1 in which said indwelling catheter comprises an endo-tracheal tube having an internal passageway and in which said elongated, flexible wand is telescopically receivable within said internal passageway of said endo-tracheal tube.

9. The apparatus as defined in claim 1 in which said source of UV-C radiation comprises a germicidal UV-C generating lamp.

10. The apparatus as defined in claim 1, further including switching means for energizing said UV generating lamp.

11. The apparatus as defined in claim 1, further including timer means for controlling the length of time that said germicidal UV-C generating lamp is energized.

12. An apparatus for exposing an indwelling catheter to germicidal ultraviolet light comprising:
(a) a hollow housing;
(b) an elongated, flexible wand connected to said hollow housing;
(c) a source of germicidal UV-C radiation carried by said hollow housing for emitting UV-C radiation in a direction toward said elongated, flexible wand; and
(d) switching means carried by said hollow housing for controllably energizing said source of germicidal UV-C radiation.

13. The apparatus as defined in claim 12 in which said apparatus further includes a UV-C-transparent disposable wand cover for covering said elongated, flexible wand.

14. The apparatus as defined in claim 12, further including timer means carried by said hollow housing for controlling the length of time that said source of germicidal UV-C radiation is energized.

15. The apparatus as defined in claim 12 in which said hollow housing is generally cylindrical in shape and in which said switching means comprises a pair of electrical contacts mounted within said housing.

16. The apparatus as defined in claim 12 in which said switching means comprises a switch member slidably connected to said housing.

17. The apparatus as defined in claim 12 in which said apparatus includes a cover for covering said elongated flexible wand and in which said apparatus further includes cover-ejecting means carried by said hollow housing for ejecting said cover.

18. The apparatus as defined in claim 17, in which said apparatus further includes safety disabling means carried by said housing for preventing energizing of said source of germicidal UV-C radiation when said wand cover is removed.

19. A method for irradiating an indwelling catheter using an apparatus comprising an elongated, flexible wand that is operably coupled with the UV-C light energy source, said method comprising the steps of positioning the elongated, flexible wand proximate the indwelling catheter and then energizing the UV-C light energy source to controllably irradiate the indwelling catheter with UV-C radiation.

20. The method as defined in claim 19 in which the apparatus comprises a timer and in which the indwelling catheter is irradiated with UV-C radiation for a selected period of time.

21. The method as defined in claim 19 in which the indwelling catheter comprises a urinary drainage catheter.

22. The method as defined in claim 19 in which the apparatus further comprises a substantially UV-transparent cover for covering the elongated, flexible wand to form a covered flexible wand and in which the method comprises the step of positioning the covered flexible wand proximate the indwelling catheter and then energizing the UV-C light energy source to controllably irradiate the indwelling catheter with UV-C radiation.

23. The method as defined in claim 19 in which the indwelling catheter comprises an endo-tracheal tube having a lumen and an exterior surface.

24. The method as defined in claim 19 in which the apparatus further comprises a timer and in which the endo-tracheal tube is irradiated with UV-C radiation for a selected period of time.

25. The method as defined in claim 24 in which said method comprises the step of inserting the elongated, flexible wand into the lumen of the endo-tracheal tube and then energizing the UV-C light energy source to controllably irradiate the lumen of the endo-tracheal tube with UV-C radiation.

26. The method as defined in claim 24 in which said method comprises the step of positioning the elongated, flexible wand proximate the exterior surface of the endo-tracheal tube and then energizing the UV-C light energy source to controllably irradiate the exterior of the endo-tracheal tube with UV-C radiation.
